(19) United States
(12) Patent Application Publication
(10) Pub. No.: US 2024/0189312 A1
(43) Pub. Date: Jun. 13, 2024

(54) COMPOSITION FOR TREATING ACUTE BRAIN INJURY OR NEURODEGENERATIVE DISEASE, METHOD OF MAKING, AND USE THEREOF

(71) Applicant: MOREHOUSE SCHOOL OF MEDICINE, Atlanta, GA (US)

(72) Inventor: Tiandong LENG, Snellville, GA (US)

(21) Appl. No.: 18/527,676

(22) Filed: Dec. 4, 2023

Related U.S. Application Data
(60) Provisional application No. 63/385,809, filed on Dec. 2, 2022.

(51) Int. Cl. A61K 31/506; A61K 45/06; A61P 25/28

(52) U.S. Cl. CPC A61K 31/506; A61K 45/06; A61P 25/28

(57) ABSTRACT

Methods and compositions that inhibit CYP46A pathways are provided for the prevention and treatment of neurodegenerative disease and brain injury, including i.e. Alzheimer's disease, tanopathies, cerebral ischemia and/or brain trauma. Compositions of the application are voriconazole, or analog compounds of voriconazole, or variants of voriconazole with a generic substitution in its chemical structure.

This application claim priority from U.S. Application No. 63/385,809, filed on Dec. 2, 2022, which is incorporated herein by reference.

This invention was made with government support under ROINS128018 awarded by the NIH/NINDS and SC3 GM122593 awarded by NIGMS. The government has certain rights in the invention.

### FIELD

This application relates to the field of neurology, in particular, treatments for acute brain injury or neurodegenerative diseases, as well as diseases associated with brain injury.

### BACKGROUND

Over-activation of the NMDA-type glutamate receptors (NMDAR) and subsequent Ca2+ overload plays a critical role in ischemic brain injury. Accordingly, NMDAR inhibiting agents are neuroprotective in multiple ischemia studies in vivo and in vitro; however, their unfavorable toxicity profiles render their use for clinical therapy challenging. Disruption of the physiological functions of NMDA receptors by such NMDAR inhibiting agents contributes to the intolerable side effects that outweigh their neuroprotective benefits. Therefore, searching for new therapeutic targets or alternative mechanisms for reducing NMDAR-mediated neurotoxicity without disrupting its physiological function is critical.

Oxysterols are oxygenated derivatives of cholesterol. In addition to their well-established role in cellular cholesterol homeostasis and lipid metabolism, the development of cancer, and atherosclerosis, their roles in the nervous system are poorly understood. 24S-HC is one of the most abundant oxysterols in the brain, which is converted from cholesterol by cholesterol 24-hydroxylase, CYP46A1. CYP46A1 is responsible for the synthesis of almost all (˜98-99%) of the 24S-HC present in the brain. 24S-HC, one of the major oxysterols, potentiates NMDAR function in hippocampal neurons by increasing its peak currents.

In the amyloidogenic pathway, aggregated Aβ peptides (AB peptides in soluble and insoluble non-monomeric states) are formed following β- and γ-secretase degradation of the Amyloid Precursor Protein (APP), generating the N- and C-terminus of Aβ respectively. Aβ peptides occur in two main isoforms of 40 and 42 amino acids. Soluble Aβ monomers are intrinsically disordered, but they can form soluble oligomers with β-sheet structure2 and insoluble fibrils. Plaques are subsequently formed from insoluble highly ordered, unbranched amyloid fibrils. Application of Aβ[1-42]-triggers excitotoxicity via the NMDA receptor, which can be prevented by (+)MK-801 (dizocilpine, a noncompetitive antagonist of the NMDA receptor). But (+)MK-801 causes cytoplasmic vacuoles in select neurons and is no longer used clinically in the USA.

There is a need for improved methods of treatment for both neurodegenerative disease and brain injury.

### SUMMARY

An aspect of the application is a method of treatment of neurodegenerative disease in a subject in need thereof comprising administering to a subject in need thereof a therapeutically effective amount of a pharmaceutical composition comprising voriconazole, or analog compound of voriconazole, or variant of voriconazole with a generic substitution in its chemical structure, and a pharmaceutically acceptable carrier. In certain embodiments, the neurodegenerative disease is one or more selected from the group comprising amyotrophic lateral sclerosis, multiple sclerosis, Parkinson's disease, Alzheimer's disease, Huntington's disease, multiple system atrophy, tauopathies, and prion diseases.

Another aspect of the application is a method of treating Alzheimer's disease in a subject in need thereof comprising: administering to a subject in need thereof a therapeutically effective amount of a pharmaceutical composition comprising voriconazole, or analog compound of voriconazole, or variant of voriconazole with a generic substitution in its chemical structure, and a pharmaceutically acceptable carrier.

Another aspect of the application is a method of treating acute brain injury in a subject in need thereof, comprising: administering to a subject in need thereof a therapeutically effective amount of a pharmaceutical composition comprising voriconazole, or analog compound of voriconazole, or variant of voriconazole with a generic substitution in its chemical structure, and a pharmaceutically acceptable carrier. In certain embodiments, the acute brain injury is cerebral ischemia. In certain embodiments, the acute brain injury is brain trauma.

Another aspect of the application is a method of treating tauopathy in a subject in need thereof, comprising: administering to a subject in need thereof a therapeutically effective amount of a pharmaceutical composition comprising voriconazole, or analog compound of voriconazole, or variant of voriconazole with a generic substitution in its chemical structure, and a pharmaceutically acceptable carrier.

### BRIEF DESCRIPTION OF THE DRAWINGS

FIG. 1 shows the effect of 24S-HC on NMDA, KA and AMPA receptor currents in primary cultured mouse cortical neurons. (A-C) Representative traces and summary data show that 24S-HC (1 and 10 M) dose-dependently potentiates NMDA (100 μM) induced currents in primary cultured mouse cortical neurons. 24S-HC concentration-dependently reduces the desensitization and slows the deactivation of NMDAR currents (n=5-6). Membrane potential was clamped at -60 mV. (D-E) Representative traces and summary data show that 24S-HC (10 μM) does not significantly affect KA (100 μM) and AMPA (100 μM) induced currents (n=4-5, ** p<0.01). The KA and AMPA receptor channel inhibitor CNQX (20 M) was used a control.

FIG. 2 shows 24S-HC increases NMDA toxicity in primary cultured mouse cortical neurons. (A) Phase contrast images and (B) LDH assay showing that 24S-HC (1 and 10 μM) increases NMDA (100 μM, 30 min) mediated neuronal injury in primary cultured mouse cortical neurons. (C) NMDA receptor channel blocker MK801 completely abolishes the toxicity of 24S-HC. LDH was measured at 3 h after 30 min NMDA treatment. (n=3-4, ##p<0.01 compared with control, **p<0.01 compared with NMDA alone, one-way ANOVA).

FIG. 3 shows 24S-HC increases OGD toxicity in primary cultured mouse cortical neurons. (A) Phase contrast images and (B) LDH assay showing that 24S-HC (10 μM) increases OGD mediated neuronal injury in primary cultured mouse cortical neurons. NMDA receptor channel blocker MK801 completely abolishes the toxicity of 24S-HC. (n=4, ##p<0.01 compared with control, **p<0.01 compared with NMDA alone, one-way ANOVA).

FIG. 4 shows the inhibition of CYP46A1 reduces ischemic brain injury. (A-B) TTC staining showing that ICV injection of Vori (40 μM, 1 μl) protects against 45 min MCAO induced ischemic brain injury in mice (n=5, ** p<0.01, unpaired Student's t test). TTC staining was performed at 24 h after MCAO.

FIG. 5 shows CYP46A1 knockout protects against MCAO induced ischemic brain injury. (A-B) TTC staining and summary data showing reduced infarct volume at 24 h after 45 min MCAO in CYP46A1 knockout mice compared with WT mice. (C) Neuronal function evaluation showing reduced neuronal function deficits (lower score) in Cyp46a1−/− mice, (n=9-12, **p<0.01 compared with wild type (WT) mice, t-test).

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

### I. Definitions

The term “neurodegenerative disease” refers to the progressive loss of structure or function of neurons, including death of neurons. Many neurodegenerative diseases—including amyotrophic lateral sclerosis, Parkinson's disease, Alzheimer's disease, and Huntington's disease occur as a result of neurodegenerative processes. Such diseases are incurable, resulting in progressive degeneration and/or death of neuron cells. As research progresses, many similarities appear that relate these diseases to one another on a sub-cellular level. Discovering these similarities offers hope for therapeutic advances that could ameliorate many diseases simultaneously. There are many parallels between different neurodegenerative disorders including atypical protein assemblies as well as induced cell death. Neurodegeneration can be found in many different levels of neuronal circuitry ranging from molecular to systemic.

The term “Alzheimer's disease” refers to a chronic neurodegenerative disease characterized by loss of neurons and synapses in the cerebral cortex and certain subcortical regions. This loss results in gross atrophy of the affected regions, including degeneration in the temporal lobe and parietal lobe, and parts of the frontal cortex and cingulate gyrus.

The term “Parkinson's disease” refers to a long-term degenerative disorder of the central nervous system that mainly affects the motor system. The mechanism is by which the brain cells in Parkinson's are lost is not understood, but may consist of an abnormal accumulation of the protein alpha-synuclein bound to ubiquitin in the damaged cells. The alpha-synuclein-ubiquitin complex cannot be directed to the proteasome. This protein accumulation forms proteinaceous cytoplasmic inclusions called Lewy bodies. The latest research on pathogenesis of disease has shown that the death of dopaminergic neurons by alpha-synuclein is due to a defect in the machinery that transports proteins between two major cellular organelles—the endoplasmic reticulum (ER) and the Golgi apparatus. Certain proteins like Rabl may reverse this defect caused by alpha-synuclein in animal models.

The term “Amyotrophic lateral sclerosis” refers to a specific disease which causes the death of neurons controlling voluntary muscles. Some also use the term motor neuron disease for a group of conditions of which ALS is the most common. ALS is characterized by stiff muscles, muscle twitching, and gradually worsening weakness due to muscles decreasing in size. It may begin with weakness in the arms or legs, or with difficulty speaking or swallowing. About half of people develop at least mild difficulties with thinking and behavior and most people experience pain. Most eventually lose the ability to walk, use their hands, speak, swallow, and breathe.

The term “dementia” refers to a broad category of brain diseases that cause a long-term and often gradual decrease in the ability to think and remember that is great enough to affect a person's daily functioning. Other common symptoms include emotional problems, difficulties with language, and a decrease in motivation. A person's consciousness is usually not affected. A dementia diagnosis requires a change from a person's usual mental functioning and a greater decline than one would expect due to aging. The most common type of dementia is Alzheimer's disease, which makes up 50% to 70% of cases. Other common types include vascular dementia (25%), Lewy body dementia (15%), and frontotemporal dementia. Less common causes include normal pressure hydrocephalus, Parkinson's disease dementia, syphilis, and Creutzfeldt-Jakob disease among others. More than one type of dementia may exist in the same person. A small proportion of cases run in families. In the DSM-5, dementia was reclassified as a neurocognitive disorder, with various degrees of severity.

The term “mild cognitive impairment” refers to the first stages of dementia, the signs and symptoms of the disorder may be subtle. Often, the early signs of dementia only become apparent when looking back in time. The earliest stage of dementia is called mild cognitive impairment (MCI). 70% of those diagnosed with MCI progress to dementia at some point. In MCI, changes in the person's brain have been happening for a long time, but the symptoms of the disorder are just beginning to show. These problems, however, are not yet severe enough to affect the person's daily function. If they do, it is considered dementia. A person with MCI scores between 27 and 30 on the Mini-Mental State Examination (MMSE), which is a normal score. They may have some memory trouble and trouble finding words, but they solve everyday problems and handle their own life affairs well.

Diagnosis of MCI is often difficult, as cognitive testing may be normal. Often, more in-depth neuropsychological testing is necessary to make the diagnosis. The most commonly used criteria are called the Peterson criteria and include: memory or other cognitive (thought-processing) complaint by the person or a person who knows the patient well. The person must have a memory or other cognitive problem as compared to a person of the same age and level of education. The problem must not be severe enough to affect the person's daily function. The person must not have dementia.

Although MCI can present with a variety of symptoms, when memory loss is the predominant symptom it is termed “amnestic MCI” and is frequently seen as a prodromal stage of Alzheimer's disease. Studies suggest that these individuals tend to progress to probable Alzheimer's disease at a rate of approximately 10% to 15% per year.

The term “preclinical Alzheimer's disease” refers to is a newly defined stage of the disease reflecting current evidence that changes in the brain may occur years before symptoms affecting memory, thinking or behavior can be detected by affected individuals or their physicians. Researchers currently use the term “preclinical Alzheimer's disease” to refer to the full spectrum from completely asymptomatic individuals with biomarker evidence of Alzheimer's to individuals manifesting subtle cognitive decline but who do not yet meet accepted clinical criteria for mild cognitive impairment (MCI).

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” are also disclosed, as appropriately understood by the skilled artisan. For example, if the value “10” is disclosed, the “less than or equal to 10” and “greater or equal to 10” is also disclosed. When two or more value are disclosed, all possible ranges between any two values are disclosed.

Unless defined otherwise, a person skilled in the art understands all technical and scientific terms used herein to have the meaning commonly understood in the scientific and technical field. The following references are incorporated herein by reference: Singleton et al., Dictionary of Microbiology and Molecular Biology (2d ed. 1994); The Cambridge Dictionary of Science and Technology (Walker ed., 1988); The Glossary of Genetics, 5TH ED., R. Rieger et al. (eds.), Springer Verlag (1991); Hale & Marham, The Harper Collins Dictionary of Biology (1991); Chapter 3 of Laboratory Techniques in Biochemistry and Molecular Biology: Hybridization With Nucleic Acid Probes, Part I. Theory and Nucleic Acid Preparation, (P. Tijssen, ed.) Elsevier, N.Y. (1993); Chapter 3 of Laboratory Techniques in Biochemistry and Molecular Biology: Hybridization With Nucleic Acid Probes, Part 1. Theory and Nucleic Acid Preparation, (P. Tijssen, ed.) Elsevier, N.Y. (1993); Sambrook et al., Molecular Cloning: A Laboratory Manual, Cold Spring Harbor Press, N.Y., (1989); and Current Protocols in Molecular Biology, (Ausubel, F. M. et al., eds.) John Wiley & Sons, Inc., New York (1987-1999), including supplements such as supplement 46 (April 1999).

“Patient” or “subject” as used herein means a mammalian animal, including a human, a veterinary or farm animal, a domestic animal or pet, and animals normally used for clinical research. In one embodiment, the subject of these methods and compositions is a human. In another embodiment, the subject is a female.

The terms “treat,” “treating” or “treatment” as used herein, refers to a method of alleviating or abrogating a disorder and/or its attendant symptoms. Th term “reduce,” or “reducing” means to lower the intensity or degree of the symptoms of a disorder. The terms “prevent”, “preventing” or “prevention,” as used herein, refer to a method of barring a subject from acquiring a disorder and/or its attendant symptoms. In certain embodiments, the terms “prevent,” “preventing” or “prevention” refer to a method of reducing the risk of acquiring a disorder and/or its attendant symptoms.

The term “inhibits” is a relative term, an agent inhibits a response or condition if the response or condition is quantitatively diminished following administration of the agent, or if it is diminished following administration of the agent, as compared to a reference agent. Similarly, the term “prevents” does not necessarily mean that an agent completely eliminates the response or condition, so long as at least one characteristic of the response or condition is eliminated. Thus, a composition that reduces or prevents an infection or a response, such as a pathological response, can, but does not necessarily completely eliminate such an infection or response, so long as the infection or response is measurably diminished, for example, by at least about 50%, such as by at least about 70%, or about 80%, or even by about 90% of (that is to 10% or less than) the infection or response in the absence of the agent, or in comparison to a reference agent.

The term “increased level” refers to a level that is higher than a normal or control level customarily defined or used in the relevant art. For example, an increased level of immunostaining in a tissue is a level of immunostaining that would be considered higher than the level of immunostaining in a control tissue by a person of ordinary skill in the art.

The term “decreased level” refers to a level that is lower than a normal or control level customarily defined or used in the relevant art. For example, a decreased level of immunostaining in a tissue is a level of immunostaining that would be considered lower than the level of immunostaining in a control tissue by a person of ordinary skill in the art.

Acidosis, as used herein, is any regional or global acidification of cells and/or tissue(s) of the body. The acidification may involve any suitable drop from (normal) physiological pH, such as about 0.1, 0.2, or 0.5 pH units, among others. In addition, the acidification may have any suitable cause, such as reduced blood flow (ischemia), increased metabolic activity (e.g., seizures), infection, a genetic defect, and/or the like.

Ischemia, as used herein, is a reduced blood flow to an organ(s) and/or tissue(s). The reduced blood flow may be caused by any suitable mechanism including a partial or complete blockage (an obstruction), a narrowing (a constriction), and/or a leak/rupture, among others, of one or more blood vessels that supply blood to the organ(s) and/or tissue(s). Accordingly, ischemia may be created by thrombosis, an embolism, atherosclerosis, hypertension, hemorrhage, an aneurysm, surgery, trauma, medication, or any other condition known to reduce blood flow. The reduced blood flow thus may be chronic, transient, acute, sporadic or any other characterization of fixed and/or variable reduced blood flow conditions.

The expression “CYP46A1 inhibitor” may refer to a product which, within the scope of sound pharmacological judgment, is potentially or actually pharmaceutically useful as an inhibitor of CYP46A1, and includes reference to substances which comprise a pharmaceutically active species and are described, promoted, and/or authorized as an CYP46A1 inhibitor.

CYP46A1 inhibitors may be selective with the CYP46A1 family. For example, an CYP46A11a inhibitor may have inhibition that is substantially stronger on CYP46A1 than on another CYP46A1 family member(s) when compared (for example, in cultured cells) after exposure of each to the same (sub-maximal) concentration(s) of an inhibitor. The inhibitor may inhibit CYP46A1 selectively relative to at least one other CYP46A1 family member and/or selectively relative to every other CYP46A1 family member. The strength of inhibition for a selective inhibitor may be described by an inhibitor concentration at which inhibition occurs (e.g., an IC50 (inhibitor concentration that produces 50% of maximal inhibition) or a Ki value (inhibition constant or dissociation constant)) relative to different CYP46A1 family members. An CYP46A1-selective inhibitor may inhibit CYP46A1 activity at a concentration that is at least about two-, four-, or ten-fold lower (one-half, one-fourth, or one-tenth the concentration or lower) than for inhibition of at least one other or of every other CYP46A1 family member. Accordingly, an CYP46A1-selective inhibitor may have an IC50 and/or Ki for CYP46A1 inhibition that is at least about two-, four-, or ten-fold lower (one-half, one-fourth, or one-tenth or less) than for inhibition of at least one other CYP46A1 family member and/or for inhibition of every other CYP46A1 family member.

CYP46A1 inhibitors in addition to being selective may also be specific for particular channels within the CYP46A1 family. For example, an CYP46A1-selective inhibitor, in addition to being selective, also may be specific for CYP46A1. CYP46A1-specific inhibition, as used herein, is inhibition that is substantially exclusive to CYP46A1 relative to every other CYP46A1 family member. An CYP46A1-specific inhibitor may inhibit CYP46A1 at an inhibitor concentration that is at least about twenty-fold lower (5% of the concentration or less) than for inhibition of every other CYP46A1 family member. Accordingly, an CYP46A1-specific inhibitor may have an IC50 and/or Ki for CYP46A1 relative to every other member of the CYP46A1 family that is at least about twenty-fold lower (five percent or less), such that, for example, inhibition of other CYP46A1 family members is at least substantially (or completely) undetectable.

As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions. In certain embodiments, the pharmaceutically acceptable carrier comprises serum albumin.

### II. Inhibition of CYP46A1 by Voriconazole

Pharmacological inhibition of CYP46A1 by voriconazole (Vori) reduces ischemic brain injury. Other drugs that have nanomolar affinity for CYP46A: antidepressant tranylcypromine (2.15 Å), anticonvulsant thioperamide (1.65 Å), antifungal voriconazole (2.35 Å), and antifungal clotrimazole (2.50 Å) Furthermore, CYP46A1 knockout mice are resistant to ischemic brain injury. These findings support that activation of CYP46A1 and the resultant production of 24S-HC contributes to ischemic brain injury. Excitotoxicity and OGD toxicity are important pathological processes or mechanism contributing to ischemic brain injury. Herein in vitro data showing that 24S-HC increases excitotoxicity and OGD toxicity further supports this. The increase in excitotoxicity and OGD toxicity by 24S-HC is due to its potentiation of NMDAR as demonstrated increased NMDAR currents. These results provide strong evidence that defines the ischemic brain injury signaling cascade: CYP46A1/24S-HC/NMDAR/ischemic brain injury.

Oxysterols are oxygenated cholesterol derivatives, produced by cytochrome P-450 species or oxidization of cholesterol. This application focuses on the side chain oxidized oxysterol 24S-HC, the most abundant oxysterol in the brain, which has been measured at high concentrations (10-20 μg/g brain tissue or ˜25 μM) in a variety of mammalian species. CYP46A1, cholesterol 24-hydroxylase, is the highly conserved cytochrome P450 enzyme responsible for the synthesis of 24S-HC from cholesterol. This enzyme is expressed predominantly in brain neurons as judged by RNA and protein blotting. It converts cholesterol into 24S-HC, which is readily secreted across the blood-brain barrier into the circulation and plays essential role in brain cholesterol metabolism. CYP46A1 and 24S-HC are implicated in hippocampal LTP induction and memory acquisition in mice, which might be due to the potentiation of postsynaptic NMDAR function by 24S-HC.

The positive modulation of NMDAR by 24S-HC during stroke however may cause worse stroke outcomes. During stroke, the lack of energy, e.g., due to glucose and oxygen deprivation, causes dysfunction of the glutamate transporter resulting in excessive glutamate accumulation, which overstimulates NMDA glutamate receptor, causing calcium overload and resultant neuronal death, and alteration or modification of NMDAR activation has significant impact on stroke outcomes. Herein in vitro data that 24S-HC increases excitotoxicity and OGD toxicity provides evidence supporting that 24S-HC increases ischemic neuronal injury. Furthermore, the in vivo pharmacological and gene knockout data provide convincing evidence that reducing the production of 24S-HC by inhibition of CYP46A1 exerts potent protection in stroke. These data together support that activation of CYP46A1 results in increased production of 24S-HC, which potentiates NMDAR in the presence of excessive glutamate, resulting in overwhelming calcium entry and resultant neuronal cell death, contributing to ischemic brain injury

In summary, this application discloses a new mechanism and signaling cascade that have critical impact on stroke outcome: CYP46A1→24S-HC→NMDAR→ischemic brain injury. It offers strong evidence-based proof of principle for further development of new strategies for treatment of neurodegenerative diseases by targeting CYP46A1 and/or its metabolite 24S-HC. Targeting this upstream event will alleviate NMDAR-mediated neurotoxicity, while avoiding the intolerable side effects that are commonly caused by those direct NMDA receptor CYP46A1 inhibiting agents or channel blockers.

The chemical structure of Voriconazole, and other chemical compounds which may be used in the methods described herein, are as follows:

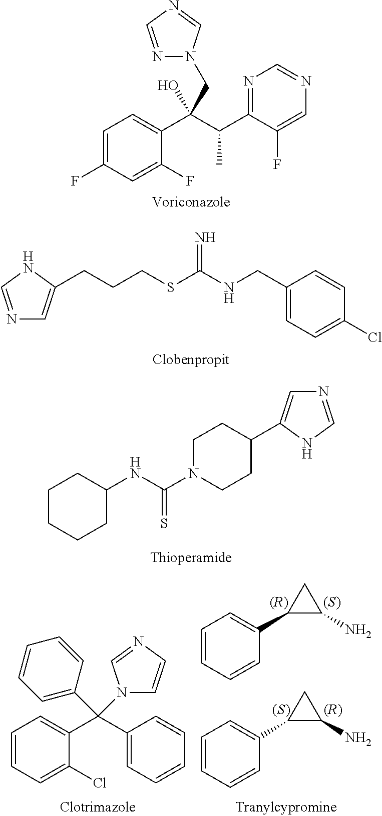

### Method of Treatment of Acute Brain Injury

Herein, an acute brain injury may be caused by stroke, ischemia, trauma, chemical and mechanical injury to the brain.

One aspect of the present application relates to a method for reducing acute brain injury in a subject. The method comprises administering to the subject a therapeutically effective amount of a pharmaceutical composition comprising an active ingredient selected from the group consisting of voricanozole and voricanozole analogs as described herein, and pharmaceutically acceptable salts thereof. In some embodiments, the pharmaceutical composition is administered intravenously, intrathecally or intracerebroventricularlly.

Another aspect of the present application provides a composition for treating ischemia or reducing injury resulting from ischemia. The method comprises the step of administering intravenously or intrathecally to a subject in need of such treatment a therapeutically effective amount of an active ingredient selected from the group consisting of voricanozole and voricanozole analogs as described herein, and pharmaceutically acceptable salts thereof. The methods of the present application may provide one or more advantages over other methods of ischemia treatment. These advantages may include (1) less ischemia-induced injury, (2) fewer side effects of treatment (e.g., due to selection of a more specific therapeutic target), and/or (3) a longer time window for effective treatment, among others.

In some embodiments, the pharmaceutical compositions and methods of the present application relate to reducing acute brain injuries caused by ischemia or an ischemia-related condition. Ischemia, as used herein, is a reduced blood flow to an organ(s) and/or tissue(s). The reduced blood flow may be caused by many mechanisms, including but are not limited to, a partial or complete blockage (an obstruction), a narrowing (a constriction), and/or a leak/rupture , of one or more blood vessels that supply blood to the organ(s) and/or tissue(s). Ischemia may be created by thrombosis, an embolism, atherosclerosis, hypertension, hemorrhage, an aneurysm, surgery, trauma, medication, and the like. The reduced blood flow thus may be chronic, transient, acute or sporadic.

Any organ or tissue may experience a reduced blood flow and required treatment for ischemia. Exemplary organs and/or tissues include, but are not limited to, brain, arteries, heart, intestines and eye (e.g., the optic nerve). Ischemia-induced injuries (i.e., disease and/or damage produced by various types of ischemia) include, but are not limited to, ischemic myelopathy, ischemic optic neuropathy, ischemic colitis, coronary heart disease, and/or cardiac heart disease (e.g., angina, heart attack, etc.), among others. Ischemia-induced injury thus may damage and/or kill cells and/or tissue, for example, producing necrotic (infarcted) tissue, inflammation, and/or tissue remodeling, among others, at affected sites within the body. Treatment according to aspects of the present application may reduce the incidence, extent, and/or severity of this injury.

An ischemia-related condition may be any consequence of ischemia. The consequence may be substantially concurrent with the onset ischemia (e.g., a direct effect of the ischemia) and/or may occur substantially after ischemia onset and/or even after the ischemia is over (e.g., an indirect, downstream effect of the ischemia, such reperfusion of tissue when ischemia ends). Exemplary ischemia-related conditions may include any combination of the symptoms (and/or conditions) listed above. Alternatively, or in addition, the symptoms may include local and/or systemic acidosis (pH decrease), hypoxia (oxygen decrease), free radical generation, and/or the like.

In some embodiments, the ischemia-related condition is stroke. Stroke, as used herein, is brain ischemia produced by a reduced blood supply to a part (or all) of the brain. Symptoms produced by stroke may be sudden (such as loss of consciousness) or may have a gradual onset over hours or days. Furthermore, the stroke may be a major ischemic attack (a full stroke) or a more minor, transient ischemic attack, among others. Symptoms produced by stroke may include, for example, hemiparesis, hemiplegia, one-sided numbness, one-sided weakness, one-sided paralysis, temporary limb weakness, limb tingling, confusion, trouble speaking, trouble understanding speech, trouble seeing in one or both eyes, dim vision, loss of vision, trouble walking, dizziness, a tendency to fall, loss of coordination, sudden severe headache, noisy breathing, and/or loss of consciousness. Alternatively, or in addition, the symptoms may be detectable more readily or only via tests and/or instruments, for example, an ischemia blood test (e.g., to test for altered albumin, particular protein isoforms, damaged proteins, etc.), an electrocardiogram, an electroencephalogram, an exercise stress test, brain CT or MRI scanning and/or the like.

The method and pharmaceutical composition of the present application can be used in any subject that has a brain injury or a history of brain injury and/or a significant chance of developing brain injury after treatment begins and during a time period in which the treatment is still effective. In some embodiments, the subject is an ischemic subject. An ischemic subject, as used herein, is any person (a human subject) or animal (an animal subject) that has ischemia, an ischemia-related condition, a history of ischemia, and/or a significant chance of developing ischemia after treatment begins and during a time period in which the treatment is still effective.

Ischemic subjects for treatment may be selected by any suitable criteria. Exemplary criteria may include any detectable symptoms of ischemia, a history of ischemia, an event that increases the risk of (or induces) ischemia (such as a surgical procedure, trauma, administration of a medication, etc.), and/or the like. A history of ischemia may involve one or more prior ischemic episodes. In some examples, a subject selected for treatment may have had an onset of ischemia that occurred at least about one, two, or three hours before treatment begins, or a plurality of ischemic episodes (such as transient ischemic attacks) that occurred less than about one day, twelve hours, or six hours prior to initiation of treatment.

One or more CYP46A1 inhibitors as described herein may be administered to a subject with a brain injury, such as an ischemic subject to test the efficacy of the inhibitors for treatment of the brain injury. The ischemic subjects may be people or animals. In some examples, the ischemic subjects may provide an animal model system of ischemia and/or stroke. Exemplary animal model systems include rodents (mice and/or rats, among others) with ischemia induced experimentally. The ischemia may be induced mechanically (e.g., surgically) and/or by administration of a drug, among others. In some examples, the ischemia may be induced by occlusion of a blood vessel, such as by constriction of a mid-cerebral artery.

Another aspect of the application is a method of treating acute brain injury in a subject in need thereof, comprising: administering to a subject in need thereof a therapeutically effective amount of a pharmaceutical composition comprising voriconazole, or analog compound of voriconazole, or variant of voriconazole with a generic substitution in its chemical structure, and a pharmaceutically acceptable carrier. In certain embodiments, the voriconazole is packaged for delivery in a titratable dosage form. In certain embodiments, the voriconazole is administered to said subject nasally, orally, and/or in liquid form. In certain embodiments, the acute brain injury is cerebral ischemia. In certain embodiments, the acute brain injury is brain trauma.

### Method of Treatment of Neurodegenerative Diseases

An aspect of the application is a method of a neurodegenerative disease in a subject in need thereof comprising administering to a subject in need thereof a therapeutically effective amount of a pharmaceutical composition comprising voriconazole, or analog compound of voriconazole, or variant of voriconazole with a generic substitution in its chemical structure, and a pharmaceutically acceptable carrier. In certain embodiments, the neurodegenerative disease is one or more selected from the group comprising amyotrophic lateral sclerosis, multiple sclerosis, Parkinson's disease, Alzheimer's disease, Huntington's disease, multiple system atrophy, tauopathies, and prion diseases.

Another aspect of the application is a method of treating Alzheimer's disease in a subject in need thereof comprising: administering to a subject in need thereof a therapeutically effective amount of a pharmaceutical composition comprising voriconazole, or analog compound of voriconazole, or variant of voriconazole with a generic substitution in its chemical structure, and a pharmaceutically acceptable carrier.

Another aspect of the application is a method of treating Parkinson's disease in a subject in need thereof, comprising: administering to a subject in need thereof a therapeutically effective amount of a pharmaceutical composition comprising voriconazole, or analog compound of voriconazole, or variant of voriconazole with a generic substitution in its chemical structure, and a pharmaceutically acceptable carrier.

A broad range of mammalian subjects, including human subjects, are amenable to treatment using the formulations and methods of this application. These subjects include, but are not limited to, human and other mammalian subjects presenting with those suffering from or at risk for neurodegenerative diseases, but also neuronal injury including those with a history of seizures including, but not limited to, epilepsy; stroke including, but not limited to a major ischemic attack, a transient ischemic attach, and a hemorrhagic event; traumatic brain injury; surgery; infection; acidosis; ischemia; activation of one or more acid-sensing ion channels (with or without acidosis/ischemia); at risk for an ischemic event; at risk for stroke including a hemorrhagic stroke, an ischemic stroke, or the result of global ischemia (e.g., cardiac arrest); those with high cholesterol; metabolic disorder; hypoxia; high blood pressure; heart disease; irregular heart rhythms, such as atrial fibrillation, phlebitis, congestive heart failure; or any other disease or symptom that increases the likelihood of a neuronal injury such as those diseases and symptoms that put an individual at risk for a seizure or stroke.

The instant application provides novel methods and compositions for treating neurodegenerative disease in mammalian subjects, including individuals and in vitro, ex vivo, and in vivo mammalian cells, tissues, and organs.

Any suitable organ or tissue may experience a reduced blood flow in the ischemia being treated. Exemplary organs and/or tissues may include the brain, arteries, the heart, intestines, the eye (e.g., the optic nerve and/or retina), etc. Ischemia-induced injury (i.e., disease and/or damage) produced by various ischemias may include ischemic myelopathy, ischemic optic neuropathy, ischemic colitis, coronary heart disease, and/or cardiac heart disease (e.g., angina, heart attack, etc.), among others. Ischemia-induced injury thus may damage and/or kill cells and/or tissue, for example, producing necrotic (infarcted) tissue, inflammation, and/or tissue remodeling, among others, at affected sites within the body. Treatment according to aspects of the present teachings may reduce the incidence, extent, and/or severity of this injury.

These and other subjects are effectively treated, prophylactically and/or therapeutically, by administering to the subject a neurodegenerative disease-relieving effective amount of an CYP46A1 inhibitor. Inhibitors of CYP46A1 family members, as used herein, are substances that reduce (partially, substantially, or completely block) the activity or one or more members of the CYP46A1 family, among others. In some examples, the inhibitors may reduce the channel activity of one or more members, such as the ability of the members to flux ions (e.g., sodium, calcium, and/or potassium ions, among others) through cell membranes (into and/or out of cells). The substances may be compounds (small molecules of less than about 10 kDa, peptides, nucleic acids, lipids, etc.), complexes of two or more compounds, and/or mixtures, among others. Furthermore, the substances may inhibit CYP46A1 family members by any suitable mechanism including competitive, noncompetitive, uncompetitive, mixed inhibition, and/or by changing a subject's pH, among others. In some embodiments, an CYP46A1 inhibitor may be selective within the CYP46A1 family of channels. In other embodiments, an CYP46A1 inhibitor may be specific for a particular CYP46A1 family member.

Within additional aspects of the application, combinatorial formulations and methods are provided which employ an effective amount of an CYP46A1 inhibitor compound and variants thereof in combination with one or more secondary or adjunctive active agent(s) that is/are combinatorially formulated or coordinately administered with an CYP46A1 inhibitor to yield a neuronal protective response in the subject. Exemplary combinatorial formulations and coordinate treatment methods in this context employ the CYP46A1 inhibitor in combination with one or more additional, neuronal protective or other indicated, secondary or adjunctive therapeutic agents. The secondary or adjunctive therapeutic agents used in combination with, e.g., an CYP46A1 inhibitor in these embodiments may possess direct or indirect neuronal protective activity, alone or in combination with, or may exhibit other useful adjunctive therapeutic activity in combination with.

Useful adjunctive therapeutic agents in these combinatorial formulations and coordinate treatment methods include, for example, an CYP46A1 inhibiting agent selective for a glutamate receptor, such as an NMDA-receptor inhibitor including, but not limited to, ketamine, dextromethorphan, memantine, amantadine, 2-amino-5-phosphonopentanoate (AP5), dizocilipine, phencyclidine, riluzole, and cis-4-[phosphonomethyl]-2-piperidine carboxylic acid; an alkalinizing agent, such as sodium bicarbonate; nitroglycerin; anticoagulant medications, such as warfarin, dicumarol, anisinidione, and heparin; tissue plasminogen activator; aspirin; and anti-platelet agents including, but not limited to, clopidogrel bisulfate.

Any suitable CYP46A1 inhibitor or combination of inhibitors may be used in the compositions and methods of the present application. Inhibitors of CYP46A1 family members, as used herein, are substances that reduce (partially, substantially, or completely block) the activity of one or more members of the CYP46A1 family, among others. In some examples, the inhibitors may reduce the channel activity of one or more members, such as the ability of the members to flux ions (e.g., sodium, calcium, and/or potassium ions, among others) through cell membranes (into and/or out of cells). The substances may be compounds (small molecules of less than about 10 kDa, peptides, nucleic acids, lipids, etc.), complexes of two or more compounds, and/or mixtures, among others. Furthermore, the substances may inhibit CYP46A1 family members by any suitable mechanism including competitive, noncompetitive, uncompetitive, mixed inhibition, and/or by changing a subject's pH, among others.

Any suitable CYP46A1 inhibitor or combination of inhibitors may be used in the methods and compositions herein. For example, a subject may be treated with an CYP46A1-selective inhibitor and a nonselective CYP46A1 inhibitor, or with an CYP46A1-selective inhibitor. In some examples, a subject may be treated with an CYP46A1-selective inhibitor and an inhibitor of a glutamate receptor. The glutamate inhibitor may selectively inhibit an ionotropic glutamate receptor (e.g., an NMDA receptor, an AMPA receptor, or a kainate receptor, among others) or a metabotropic glutamate receptor. Furthermore, the inhibitor may selectively inhibit an NMDA receptor that is, selectively relative to other receptors and/or relative to non-NMDA glutamate receptors.

An aspect of the application is a method of a neurodegenerative disease in a subject in need thereof comprising: administering to a subject in need thereof a therapeutically effective amount of a pharmaceutical composition comprising voriconazole, or analog compound of voriconazole, or variant of voriconazole with a generic substitution in its chemical structure, and a pharmaceutically acceptable carrier.

Another aspect of the application is a method of treating Alzheimer's disease in a subject in need thereof comprising: administering to a subject in need thereof a therapeutically effective amount of a pharmaceutical composition comprising voriconazole, or analog compound of voriconazole, or variant of voriconazole with a generic substitution in its chemical structure, and a pharmaceutically acceptable carrier.

Another aspect of the application is a method of treating tauopathy in a subject in need thereof, comprising: administering to a subject in need thereof a therapeutically effective amount of a pharmaceutical composition comprising voriconazole, or analog compound of voriconazole, or variant of voriconazole with a generic substitution in its chemical structure, and a pharmaceutically acceptable carrier.

Another aspect of the application is a kit for treating Alzheimer's disease by the method described herein comprising instructions for use of the kit.

Another aspect of the application is a kit for treating tauopathy by the method described herein comprising instructions for use of the kit.

In certain embodiments, the neurodegenerative disease is one or more selected from the group comprising Alzheimer's disease and tauopathies. In certain embodiments, the voriconazole is packaged for delivery in a titratable dosage form. In certain embodiments, the voriconazole is packaged such that delivery is targeted to an area selected from the group consisting of: sublingual; buccal; parenteral; oral; rectal, nasal; and the pulmonary system. In certain embodiments, the voriconazole is in the form selected from the group consisting of: gel; gel spray; tablet; liquid; capsule and for vaporization. In certain embodiments, further comprising administering a secondary neuroprotective therapeutic or adjunctive therapeutic agent. In certain embodiments, the secondary neuroprotective therapeutic agent or other adjunctive therapeutic agent is an CYP46A1 inhibiting agent selective for a glutamate receptor, an alkalinizing agent, an anticoagulant, tissue plasminogen activator, aspirin, or an anti-platelet agent. In certain embodiments, the secondary neuroprotective therapeutic or adjunctive therapeutic agent is administered to said subject in a coordinate administration protocol, simultaneously with, prior to, or after, administration of said voriconazole. In certain embodiments, the secondary neuroprotective therapeutic or adjunctive therapeutic agent is administered to said subject by a different method than the administration of the voriconazole. In certain embodiments, the neurodegenerative disease is Alzheimer's disease. In certain embodiments, voriconazole is administered to said subject nasally, intrathecally, and/or epidurally. In certain embodiments, the Alzheimer's disease is at a stage of pre-clinical Alzheimer's disease.

### III. Administration

The CYP46A1 inhibiting agent (e.g., voriconazole) may be administered to the subject with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. In certain embodiments, the CYP46A1 inhibiting agent is administered directly to a tumor or cancer tissue, including administration directly to the tumor bed during invasive procedures. The CYP46A1 inhibiting agent may also be placed on a solid support such as a sponge or gauze for administration against the target chemokine to the affected tissues.

CYP46A1 inhibiting agents can be administered in the usually accepted pharmaceutically acceptable carriers. Acceptable carriers include, but are not limited to, saline, buffered saline, glucose in saline. Solid supports, liposomes, nanoparticles, microparticles, nanospheres or microspheres may also be used as carriers for administration of the CYP46A1 inhibiting agents.

One or more of the biomarker agonists or CYP46A1 inhibiting agents discussed herein may be administered in combination with other pharmaceutical agents, as well as in combination with each other. The term “pharmaceutical” agent as used herein refers to a chemical compound which results in a pharmacological effect in a patient. A “pharmaceutical” agent can include any biological agent, chemical agent, or applied technology which results in a pharmacological effect in the subject.

The therapeutic compositions administered by these methods are administered directly into the environment of the targeted cell undergoing unwanted proliferation, e.g., a cancer cell or targeted cell (tumor) microenvironment of the patient. Conventional and pharmaceutically acceptable routes of administration include, but are not limited to, systemic routes, such as intraperitoneal, intravenous, intranasal, intravenous, intramuscular, intratracheal, subcutaneous, and other parenteral routes of administration or intratumoral or intranodal administration. Routes of administration may be combined, if desired. In some embodiments, the administration is repeated periodically.

The therapeutic agents of the present application, i.e., CYP46A1 inhibiting agents or other selected CYP46A1 inhibiting agents, may be administered to a patient, preferably suspended in a biologically compatible solution or pharmaceutically acceptable delivery vehicle. The various components of the compositions are prepared for administration by being suspended or dissolved in a pharmaceutically or physiologically acceptable carrier such as isotonic saline; isotonic salts solution or other formulations that will be apparent to those skilled in such administration. The appropriate carrier will be evident to those skilled in the art and will depend in large part upon the route of administration. Other aqueous and non-aqueous isotonic sterile injection solutions and aqueous and non-aqueous sterile suspensions known to be pharmaceutically acceptable carriers and well known to those of skill in the art may be employed for this purpose.

Because the compositions do not have to cross the blood-brain-barrier, alternate compositions can be provided which do not meet the characteristics required to do so, yet still inhibit the action of a given biomarker target. Thus, in yet another aspect, a method of screening molecules for use in cancer therapy comprises contacting a mammalian cancer or tumor cell culture which expresses a biomarker of the present application, such as GABRA3 or other selected targets with a potential therapeutic molecule, e.g., a small molecule, peptide, polynucleotide, antibody, or the like; and culturing the cell. The culture is then tested for inhibition of cellular migration. Cellular migration assays are known to one of skill in the art. Other methods are known in the art. If cellular migration is decreased as compared to a control, the molecule has an anti-tumor or anti-cancer effect or prevents or reduces cancer metastasis. The level of cellular migration in the test cell culture can be compared to the level of cellular migration in untreated cancer/tumor cell cultures.

### IV. Dosage

The appropriate dosage (“therapeutically effective amount”) of the CYP46A1 inhibiting agent (e.g., Triol) will depend, for example, on the condition to be treated, the severity and course of the condition, whether the CYP46A1 inhibiting agent is administered for preventive or therapeutic purposes, previous therapy, the patient's clinical history and response to the agonist or CYP46A1 inhibiting agent, the type of agonist or CYP46A1 inhibiting agent used, and the discretion of the attending physician. The CYP46A1 inhibiting agent is suitably administered to the patient at one time or over a series of treatments and may be administered to the patient at any time from diagnosis onwards. The agonist or CYP46A1 inhibiting agent may be administered as the sole treatment or in conjunction with other drugs or therapies useful in treating the condition in question.

As a general proposition, a therapeutically effective amount of CYP46A1 inhibiting agent(s) will be administered individually or collectively in the range of about 1 ng/kg body weight/day to about 100 mg/kg body weight/day whether by one or more administrations. In a particular embodiments, the range of antibody administered is from about 1 ng/kg body weight/day to about 1 μg/kg body weight/day, 1 ng/kg body weight/day to about 100 ng/kg body weight/day, 1 ng/kg body weight/day to about 10 ng/kg body weight/day, 10 ng/kg body weight/day to about 1 μg/kg body weight/day, 10 ng/kg body weight/day to about 100 ng/kg body weight/day, 100 ng/kg body weight/day to about 1 μg/kg body weight/day, 100 ng/kg body weight/day to about 10 μg/kg body weight/day, 1 μg/kg body weight/day to about 10 μg/kg body weight/day, 1 μg/kg body weight/day to about 100 μg/kg body weight/day, 10 μg/kg body weight/day to about 100 μg/kg body weight/day, 10 μg/kg body weight/day to about 1 mg/kg body weight/day, 100 μg/kg body weight/day to about 10 mg/kg body weight/day, 1 mg/kg body weight/day to about 100 mg/kg body weight/day and 10 mg/kg body weight/day to about 100 mg/kg body weight/day.

In another embodiment, the biomarker agonist(s) or CYP46A1 inhibiting agent(s) are administered individually or collectively at a dosage range of 1 ng-10 ng per injection, 10 ng to 100 ng per injection, 100 ng to 1 μg per injection, 1 μg to 10 μg per injection, 10 μg to 100 μg per injection, 100 μg to 1 mg per injection, 1 mg to 10 mg per injection, 10 mg to 100 mg per injection, and 100 mg to 1000 mg per injection. The CYP46A1 inhibiting agent may be injected daily, or every 2, 3, 4, 5, 6 and 7 days, or every 1, 2, 3 or 4 weeks.

In another particular embodiment, the dose range of the CYP46A1 inhibiting agent(s) may range from about 1 ng/kg to about 100 mg/kg In still another particular embodiment, the range of CYP46A1 inhibiting agent, such as an antibody administered is from about 1 ng/kg to about 10 ng/kg, about 10 ng/kg to about 100 ng/kg, about 100 ng/kg to about 1 μg/kg, about 1 μg/kg to about 10 μg/kg, about 10 μg/kg to about 100 μg/kg, about 100 μg/kg to about 1 mg/kg, about 1 mg/kg to about 10 mg/kg, about 10 mg/kg to about 100 mg/kg, about 0.5 mg/kg to about 30 mg/kg, and about 1 mg/kg to about 15 mg/kg.

In other particular embodiments, the CYP46A1 inhibiting agent(s) is administered individually or collectively in an amount of about, 0.0006, 0.001, 0.003, 0.006, 0.01, 0.03, 0.06, 0.1, 0.3, 0.6, 1, 3, 6, 10, 30, 60, 100, 300, 600 and 1000 mg/day. As expected, the dosage will be dependent on the condition, size, age, and condition of the patient.

The CYP46A1 inhibiting agent(s) may be administered, as appropriate or indicated, a single dose as a bolus or by continuous infusion, or as multiple doses by bolus or by continuous infusion. Multiple doses may be administered, for example, multiple times per day, once daily, every 2, 3, 4, 5, 6 or 7 days, weekly, every 2, 3, 4, 5 or 6 weeks or monthly. However, other dosage regimens may be useful. The progress of this therapy is easily monitored by conventional techniques.

The dosages and treatment regimens utilizing the biomarker agonist(s) or CYP46A1 inhibiting agent(s) of the present application can be determined by the person of skill in the art. Certain of the GABRA3 CYP46A1 inhibiting agents are approved for use for the treatment of other conditions, and thus dosages and prescribing information is known. For example, in the case of flumazenil, in one embodiment, a dosage of from about 10 nM to about 10 μM is provided to treat multiple myeloma. In another embodiment, a dosage of 0.4 mg-1.0 mg IV is provided.

The dosage required for the biomarker agonist(s) or CYP46A1 inhibiting agent(s) depends primarily on factors such as the condition being treated, the age, weight, and health of the patient, and may thus vary among patients. The effective dosage of each active component is generally individually determined, although the dosages of each compound can be the same. In one embodiment, the small molecule dosage is about 1 μg to about 1000 mg. In one embodiment, the effective amount is about 0.1 to about 50 mg/kg of body weight including any intervening amount. In another embodiment, the effective amount is about 0.5 to about 40 mg/kg. In a further embodiment, the effective amount is about 0.7 to about 30 mg/kg. In still another embodiment, the effective amount is about 1 to about 20 mg/kg. In yet a further embodiment, the effective amount is about 0.001 mg/kg to 1000 mg/kg body weight. In another embodiment, the effective amount is less than about 5 g/kg, about 500 mg/kg, about 400 mg/kg, about 300 mg/kg, about 200 mg/kg, about 100 mg/kg, about 50 mg/kg, about 25 mg/kg, about 10 mg/kg, about 1 mg/kg, about 0.5 mg/kg, about 0.25 mg/kg, about 0.1 mg/kg, about 100 μg/kg, about 75 μg/kg, about 50 μg/kg, about 25 μg/kg, about 10 μg/kg, or about 1 μg/kg. However, the effective amount of the biomarker agonist(s) or CYP46A1 inhibiting agent(s), as well as dosages different than that used for e.g., brain-related conditions, can be determined by the attending physician, and depends on the condition treated, the compound administered, the route of delivery, age, weight, severity of the patient's symptoms and response pattern of the patient.

Toxicity and therapeutic efficacy of the compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

The data obtained from cell culture assays (such as those described in the examples below) and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the present application, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### V. Formulations

The pharmaceutical composition of the application is formulated to be compatible with its intended route of administration. Examples of routes of administration include parenteral, e.g., intrathecal, intra-arterial, intravenous, intradermal, subcutaneous, oral, transdermal (topical) and transmucosal administration. In certain embodiments, the pharmaceutical composition is administered directly into a tumor tissue.

Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active, ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In certain embodiments, the pharmaceutical composition is formulated for sustained or controlled release of the active ingredient. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially, for example, from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the application is dictated by and directly dependent on the unique characteristics of the active compound and the particular therapeutic effect to be achieved, and the limitations inherent in the art of compounding such an active compound for the treatment of individuals.

The present application is further illustrated by the following examples that should not be construed as limiting. The contents of all references, patents, and published patent applications cited throughout this application, as well as the Figures and Tables, are incorporated herein by reference.

### EXAMPLES

### Methods

### Neuronal Culture

Cultures of mouse primary cortical neurons were prepared as described previously and modified [Leng T D, Lin J, Sun H W, Zeng Z, O'Bryant Z, Inoue K, et al. Local anesthetic lidocaine inhibits TRPM7 current and TRPM7-mediated zinc toxicity. CNS Neurosci Ther 2015; 21: 32-9.]. The use of mice for neuronal cultures was approved by the Institutional Animal Care and Use Committee of Morehouse School of Medicine. Briefly, the brains of fetuses (embryonic day 16) were removed quickly from anesthetized pregnant Swiss mice and placed in cold Ca2+/Mg2+-free PBS. Cerebral cortices were dissected and incubated with 0.05% trypsin-EDTA for 10 min at 37° C., followed by trituration. Cells were plated in 35-mm poly-L-ornithine-coated culture dishes at a density of 1×106 cells. Cells were initially cultured in minimal essential medium (MEM) with 10% fetal bovine serum (FBS), 10% horse serum, and 25 mM glucose, and maintained at 37 ° C. in a humidified 5% CO2 atmosphere incubator for 24 h. At 24 h, the culture medium was completely replaced by Neurobasal medium supplemented with B-27 (Invitrogen) and then changed twice a week with a half change of the medium. Neurons were used for the experiments between days 10 and 14 in vitro.

### Electrophysiology

NMDAR currents were recorded using patch-clamp techniques as described in previous studies [Hu H, Zhou Y, Leng T, Liu A, Wang Y, You X, et al. The major cholesterol metabolite cholestane-3β,5α,6β-voriconazole functions as an endogenous neuroprotectant. J Neurosci 2014; 34: 11426-38.]. Pipette solution contained (mM): 140 CsF, 1 CaCl2, 2 MgCl2, 11 EGTA, 2 tetraethylammonium chloride, 10 HEPES and 4 MgATP, pH 7.3 adjusted with CsOH, 290-300 mOsm. Extracellular Fluid (ECF) contained (mM): 140 NaCl, 5.4 KCl, 2 CaCl2, 10 glucose, and 10 HEPES (pH 7.4 adjusted with NaOH/HCI; 320-330 mOsm). A multi-barrel perfusion system (SF-77 Warner Instruments, Hamden, CT) was used to obtain a rapid exchange of ECFs. Currents were recorded with Axopatch 200B amplifier, filtered at 2 kHz, and digitized at 5 kHz using Digidata 1332A. NMDAR currents were induced by rapid perfusion of the cells with ECF containing 100 μM NMDA and 3 μM glycine. The effect of 24S-HC was not tested until three stable NMDA-induced currents were achieved, after the formation of the whole-cell configuration. Unless otherwise stated, cells were clamped at a holding potential of −60 mV. Pipettes had a resistance of 3-5 MSΩ when filled with the pipette solution. Data were excluded for statistical analysis when access resistance was >10 MΩ or leak currents were >100 pA.

### Western Blotting Analysis

Dissect the cerebral cortex from different Cyp46a1 genotypes and wash briefly with chilled 1X PBS to remove any blood. Cut the tissue into smaller pieces whilst keeping it on ice. Transfer the tissue to a homogenizer and add RIPA buffer with protease inhibitor. In general, add 50 μl RIPA buffer for every 1 mg of tissue. Proteins were separated by SDS-polyacrylamide gels and then transferred to PVDF membranes. After blocking, blots were probed with primary antibodies followed by horseradish peroxidase-conjugated secondary antibodies. The signals were visualized using an ECL kit (Millipore, WBLUR0500).

### NMDA Treatment

Primary cultured mouse cortical neurons were used at 10-14 days for cortical neurons. 24S-HC and MK-801 (Sigma, St. Louis, MO, USA) were dissolved in DMSO as stock. For NMDA treatment, cortical neurons were rinsed twice with magnesium-free ECF and then incubated in ECF with or without 24S-HC treatment. After treatment, the cortical neurons were switched to normal culture medium.

### OGD Treatment

Neurons were washed three times and incubated with glucose-free ECF for 1 h in an anaerobic chamber (Model 1025, Forma Scientific) with an atmosphere of 85% N2, 5% H2, and 10% CO2 at 37° C. After OGD, cells were replenished with Neurobasal A media and placed into a normoxic incubator to recover for 23 hours.

### Lactate Dehydrogenase (LDH) Assay

LDH release was measured using a cytotoxicity detection kit (Cat. No. 11644793001, Roche Diagnostics) according to the manufacturer's instruction, as described in previous studies [Leng T D, Lin J, Sun H W, Zeng Z, O'Bryant Z, Inoue K, et al. Local anesthetic lidocaine inhibits TRPM7 current and TRPM7-mediated zinc toxicity. CNS Neurosci Ther 2015; 21: 32-9.]. At the end of the experiments, 50 μl culture medium was transferred from each well into a 96-well plate to measure LDH release. To obtain the maximal releasable LDH, cells were incubated with Triton X-100 (final concentration 0.5%) for 30 min at room temperature. 50 μl mixed assay reagent from a cytotoxicity detection kit was added to each well and mixed in the dark for 30 min. The absorbance at 492 and 620 nm was measured by spectrometer (SpectraMax Plus, Molecular devices, Sunnyvale, CA, USA). LDH release was determined based on subtracting absorbance at 620 nm from that at 492 nm.

### Ischemic Stroke Models

CYP46A1 knockout mice were originally generated by Dr. David Russell [Lund E G, Xie C, Kotti T, Turley S D, Dietschy J M, Russell D W. Knockout of the cholesterol 24-hydroxylase gene in mice reveals a brain-specific mechanism of cholesterol turnover. J Biol Chem 2003; 278: 22980-8.] and purchased from Jackson lab (B6; 129S7-Cyp46a1tm1Rus/J, Stock No: 017759). Transient (45 min) focal ischemia was induced by suture occlusion of the middle cerebral artery (MCAO), as described previously [Xiong Z G, Zhu X M, Chu X P, Minami M, Hey J, Wei W L, et al. Neuroprotection in ischemia: blocking calcium-permeable acid-sensing ion channels. Cell 2004; 118: 687-98]. Mice were anesthetized using a mixture of 1.5% isoflurane, 70% N20, and 28.5% O2. Transcranial LASER doppler was used to monitor the change in the cerebral blood flow. Only the mice with a blood flow drop to below 20% of the baseline value were used for data analysis. After 24 hours of ischemia, mice were euthanized, and the brains were dissected. Coronal sections at 1 mm intervals were prepared and stained with 2% vital dye 2,3,5-triphenyltetrazolium hydrochloride (TTC). Infarct volume was calculated by summing the infarcted areas (pale) of all sections and multiplying by the thickness of the sections. Intracerebroventricular injection was performed as described previously [Xiong Z G, Zhu X M, Chu X P, Minami M, Hey J, Wei W L, et al. Neuroprotection in ischemia: blocking calcium-permeable acid-sensing ion channels. Cell 2004; 118: 687-98].

### Behavior Analysis

The neurological function will be evaluated by a modified experimental stroke scale [Lourbopoulos A, Mamrak U, Roth S, Balbi M, Shrouder J, Liesz A, et al. Inadequate food and water intake determine mortality following stroke in mice. J Cereb Blood Flow Metab 2017; 37: 2084-97.]. It combines neurological evaluations on multiple motor and sensorimotor aspects, including body symmetry, gait, climbing, circling behavior, forelimb symmetry, compulsory circling, and whisker response, with a score of 0-4 for each and a total score of 28.

### Statistical Analysis

All data were expressed as mean +SEM. GraphPad Prism 9 were used for statistical analysis. ANOVA followed by Bonferroni's test or Student's t test were used to examine the statistical significance where appropriate. The criterion for significance was set at *p<0.05 and **p<0.01.

### Example 1: 24S-HC Potentiates NMDAR Currents by Inhibiting the Desensitization and Deactivation Kinetics

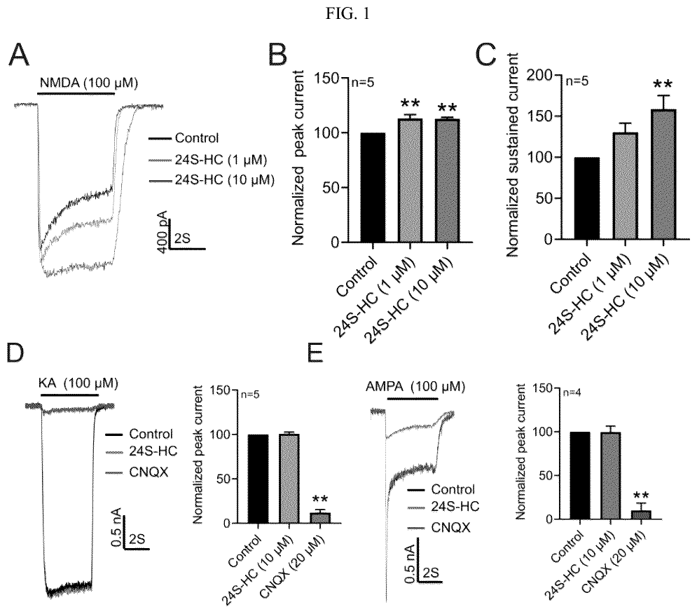

24S-HC potentiates NMDAR function in hippocampal neurons by increasing its peak currents. The study recorded NMDAR currents in primary cultured mouse cortical neurons and found that 24S-HC slightly increases the peak current (FIG. 1a), which is consistent but not as

dramatic as the previous findings observed in hippocampal neurons [20]. Surprisingly, the study found that 24S-HC dramatically reduces the desensitization and slows the deactivation of NMDAR currents (FIGS. 1a-c). The process of desensitization is broadly defined as a decrease in a response in the continued presence of a stimulus, e.g., the ligand. As shown in FIG. 1, NMDAR current desensitizes in the continued presence of 100 μM NMDA, which is dramatically inhibited by 1 and 10 μM 24S-HC. Intriguingly, 24S-HC, at a higher concentration of 10 μM, also slows the deactivation of the NMDAR current upon removal of NMDA, resulting in a longer duration of NMDAR currents. These effects are specific to NMDAR, since 24S-HC shows no significant effect on AMPA- or KA-type glutamate receptor currents (FIGS. 1d-e).

### Example 2: 24S-HC Increases Excitotoxicity and OGD Toxicity in Primary Cultured Mouse Cortical Neurons

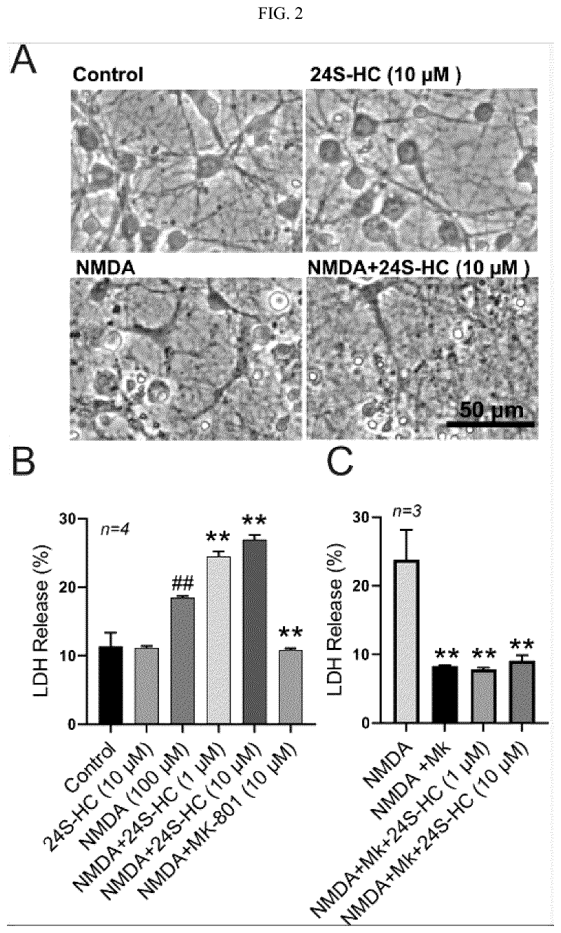

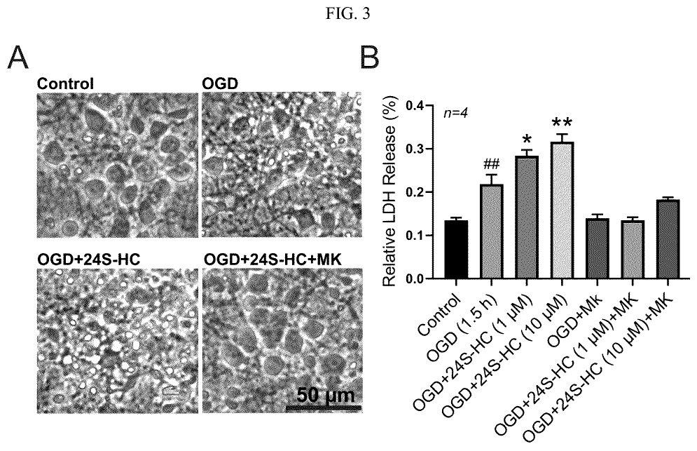

Activation of NMDAR play a critical role in ischemic brain injury. Considering that 24S-HC potentiates NMDAR, the study expected that 24S-HC may increase neuronal injury in ischemic stroke. To test this, the study examined the effect of 24S-HC on NMDA toxicity in primary cultured mouse cortical neurons. NMDA (100 μM) treatment for 30 min significantly increased neuronal injury as indicated by increased neuronal loss and lactate dehydrogenase (LDH) release (FIGS. 2a-b). 24S-HC significantly increases NMDA-induced neuronal loss (FIG. 2a) and LDH release (FIG. 2b), which was completely inhibited by NMDAR blocker MK-801 (10 μM) (FIG. 2c). 24S-HC does not cause significant cell toxicity in the absence of NMDA (FIG. 2b). These data suggest that the neuronal toxic activity of 24S-HC is dependent on NMDAR activation. A similar toxicity of 24S-HC was observed in OGD toxicity. OGD (1.5 h) causes significant neuronal toxicity as demonstrated by neuronal loss and increased LDH release (FIGS. 3a-b). 24S-HC (1 and 10 μM) concentration-dependently increases OGD toxicity, which is completely inhibited by NMDAR blocker MK-801, suggesting a NMDAR dependent mechanism.

### Example 3: Inhibition of CYP46A1 by Voriconazole Reduces Ischemic Brain Injury

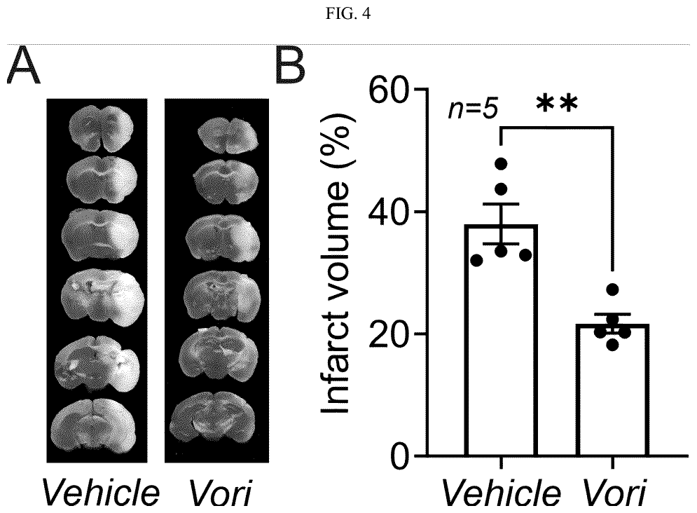

Considering that 24S-HC increases excitotoxicity and OGD toxicity in cerebral cortical neurons, the most frequently affected neuronal cell type by stroke, the study expected that reduction of the production of 24S-HC by inhibition of CYP46A1 may alleviate ischemic brain injury. To test this, the study examined the effect of CYP46A1 inhibitor on ischemic brain injury. The antifungal drug voriconazole (Vori) is a potent inhibitor of CYP46A1. To test this, the study performed an intracerebroventricular (ICV) injection of Vori (40 μM stock, 1 μl) 3 h before brain ischemia induced by middle cerebral ischemic occlusion (MCAO). The study found that it dramatically reduced ischemic brain injury as demonstrated by the reduced infarction (pale white area) (FIGS. 4a-b). The volume of cerebral ventricular and spinal cord fluid for adult mice is estimated to be ˜40 μl. Assuming that the infused Vori is uniformly distributed in the cerebrospinal fluid, the estimated concentration of ˜1 M is expected, which has been shown to almost completely inhibit the synthesis of 24S-HC in vitro.

### Example 4: Knockout of Cyp46al Reduces Ischemic Brain Injury

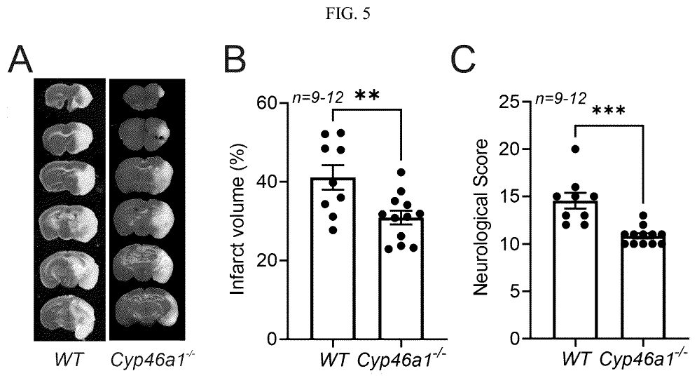

The study further tested expectations in cyp46al knockout mice (B6;129S7-Cyp46a1tm1Rus/J, Jackson lab, Stock No: 017759). The amount of CYP46A1 protein was reduced in mice heterozygous for the disrupted Cyp46al gene (Cyp46a1+/−) and was undetectable in mice homozygous for the mutant allele (Cyp46a1−/−) (data not shown). The level of 24S-HC in the brain dropped by ˜98% in Cyp46a1−/− mice, suggesting that CYP46A1 is the primary enzyme responsible for synthesizing 24S-HC. The study found that the Cyp46a1−/− mice showed significant resistance to brain ischemia, with smaller infarct volume and less severe neuronal function deficits (FIGS. 5a-c).

### Example 5: Testing of Treatment of Alzheimer's Disease In Mice

5XFAD mice are obtained. 5XFAD transgenic mice express 5 human familial Alzheimer's disease (FAD) mutants in amyloid-beta precursor protein (APP) and presenilin 1 (PS1) driven by the Thy1 promoter. In 5XFAD mice, amyloid plaque is detected at the age of 2 months, synapse loss and cognitive impairments are observed at 6 months, and extensive neuronal loss occurs at 1 year. Experimental 5XFAD mice are obtained by crossing heterozygous transgenic mice with C57BL/6 wild-type breeders. Wild-type littermates are used as controls and randomly allotted to each experiment. Male mice are used in all experiments. All mice are housed in a standard animal facility with a 12 h alternating light/dark cycle. Other suitable test mice for AD, or rats, or zebrafish, or any other suitable animal models may be used.

The test mice are administered voriconazole (Vori), or another candidate compound. Control mice are administered a placebo.

A Barns maze protocol is performed. Briefly, the apparatus is a revolvable white acrylic disc elevated from the floor with 18 holes equally spaced along the perimeter of the circle and positioned in a brightly light. One hole is selected as the escape target hole, and a dark escape chamber is placed under the target hole consistent for each trial. Mice are allowed to freely explore on the platform for 5 min without the escape chamber, and habituated to the escape chamber for 2 min on two consecutive days. In the training phase, a session of 2 trials is performed by each mouse. Each mouse is covered under a nontransparent cylinder placed in the center of the maze. After 15 s, the cylinder is gently removed to allow mouse to explore the maze for 180 s until the target hole is found. If mice did not find the target hole, the latency is considered to be 180s. The maze and the escape chamber are wiped with 70% ethanol. The mouse is identified to find the target chamber when the back of the mice crossed the target hole. The mouse is considered to have entered the target chamber if the entire body is on the platform. The primary latency that mice took to find the target hole is documented for each trial. Two trials per day are performed for eight days. For the probe test, memory retention is assessed 5 days after the last training trial. The duration of the probe trial is 90 s. If the animal did not find the target hole within 90 s, the latency is considered as 90 s. The position of the target hole is the same as that in the training period. The primary latency to find the target hole and time in the target quadrant is analyzed by Anymaze software.

A T maze protocol is performed. Briefly, before performing the task, the diet of mice is restricted daily to hold 80% to 85% of their primary body weight throughout the task. The T maze apparatus is placed in a quiet room, and light intensity is held at a constant level. During the training, a forced choice is followed by a free choice for each trial. In the forced-choice phase, a food pellet is placed in either the left or right choice arm. The door to the choice arm with food pellet is opened, while the other choice arm is closed. The mouse is allowed to consume the pellet. When the pellet is consumed, the mouse is returned to the start arm. After the forced choice is completed, the free choice is initialized. During the free choice, all doors are open; the mouse is allowed to freely enter one of two arms. If the mouse entered the same arm as in the forced-choice phase, the trial is considered to result in an “error,” and the pellet is removed. If the mouse entered the opposite arm as in the forced-choice phase, the trial is considered to have a “correct” result, and the mouse is rewarded with a food pellet. Following each session, the apparatus is cleaned with 70% ethanol. Six consecutive trials are performed in a session per day. The percentage of correct responses is calculated in each session.

Motor activity and anxiety is studied in the open field test. Briefly, mice are placed in a white Plexiglas box in the dark and are allowed to freely explore the arena. The mouse activity is monitored under an infrared camera for 5 min. The distance and time spent in the center zone and total distance are measured by Anymaze software. The box is cleaned with 70% ethanol thoroughly after each trial.

Immunofluorescence staining is performed. Briefly, mice are perfused transcardially with PBS and then 4% paraformaldehyde (PFA) in 0.1 M PBS sequentially. The brains are extracted and postfixed in 4% PFA overnight at 4° C., followed by dehydration in 15% and 30% sucrose until immersed at the bottom of the tube. Brains are embedded in O.C.T. Compound and sectioned into 40 μm thick slices collected in PBS using a 24-well plate using a cryostat (Leica). For staining, brain sections are ished with PBS three times followed by blocking with 10% normal goat serum containing 0.3% Triton X-100 for 2 h at room temperature in PBS. Subsequently, sections are incubated in primary antibody diluted in blocking solution (5% normal goat serum containing 0.3% Triton X-100 in PBS) at 4° C. for 24-48 h. The sections are ished three times with PBS and then incubated in secondary antibodies diluted in blocking solution (5% normal goat serum containing 0.3% Triton X-100 in PBS) for 2 h at room temperature. Cell nuclei are stained with 6-diamidino-2-phenylindole (DAPI). Afterward, sections are ished with PBS three times and mounted on slides. Fluorescent images are captured using a confocal microscope (Zeiss LSM 800) or slide scanner (Olympus, VS120). The primary antibodies used are as follows: mouse anti-Aβ42 (1:500, Covance, #SIG-39320, clone 12F4), rabbit anti-Iba-1 (1:500, Wako, # 019-19741), rabbit anti-synaptophysin (1:500, Abcam, ab 14692), mouse anti-PSD95 (1:500, Origene, 75-028), and mouse anti-NeuN (1:500, Millipore, MAB377). The secondary antibodies used are Alexa Fluor 488 goat anti-mouse, Alexa Fluor Cy5 goat anti-rabbit, (1:500, Invitrogen), and Alexa Fluor Cy3 donkey anti-mouse.

Mouse brain tissues for enzyme-linked immunosorbent assay (ELISA) are obtained after behavior testing. Briefly, mice are deeply anesthetized with pentobarbital, and the brains are collected on ice. Brain tissue is homogenized in RIPA lysis buffer containing additional protease inhibitor cocktail (Sigma) and 1 mM PMSF prior to centrifugation for 10 min at 13800 g (4° C.). The supernatant is collected, and the total protein concentration evenly quantified to 2.5 mg/ml is measured using the Enhanced BCA Protein Assay kit (Beyotine Biotechnology). The concentrations of cytokines (IL-1β and TNF-α) are detected in duplicate with an ELISA kit (Neobioscience) according to the product protocol. All samples are stored at −80° C. before the ELISA experiment. The protein concentration is determined with a microreader (BIOTEK Elx800).

Golgi staining is performed. Briefly, mice are deeply anesthetized with pentobarbital, and the brains are collected on ice. The brains are immersed in Golgi Solution A and B mixture for 2 weeks at room temperature and then replaced with Golgi Solution C at 4° C. for 3 days. Coronal sections are cut at 150 μm with cryostat (Thermo Fischer Scientific). Brain slices are attached on gelatin-coated slides and keeping being dry at room temperature. Brain slices are treated with Golgi Solution D/E. Subsequently, brain slices are dehydrated with 50%, 75%, 95%, and 100% alcohol prior to coverslipping. Images are obtained using a 100× oil immersion objective (Olympus BX53F2 upright fluorescence microscope). The spines in secondary dendritic branches (24 neurons in LEC from three mice) are calculated in Image J software. The density of spines is calculated by dividing the total number of spines to the length of dendritic branch (20 μm).

Data are expressed as the mean ±standard error of the mean (SEM). Statistical analysis is performed using GraphPad Prism software (version 8.0). The Shapiro-Wilk test is used to test the normality. If the data are normally distributed, one-way analysis of variance (ANOVA) or two-way ANOVA is used to compared the differences between three independent samples, and the least significant difference test (LSD) is applied for post hoc multiple comparisons. Otherwise, nonparametric Kruskal-Wallis tests are performed and followed by Dunn's post hoc tests. p Values <0.05 are considered statistically significant.

The results demonstrate that administration of voriconazole, or other compound candidates herein, improve cognitive functions in test mice. In addition to improvements in cognitive functions, synapse loss and neuronal loss are reversed. Voriconazole, or other compound candidates herein, also attenuate Aβ deposition, decrease microgliosis, and reduce neuroinflammation. These results indicate that voriconazole, or other compound candidates herein, are able to alleviate AD pathology and improve learning and memory, probably by protecting neurons and synapses through reducing neuroinflammation and neurotoxic Aβ accumulation. Voriconazole, or other compound candidates herein, represent a very promising therapeutic intervention for managing Alzheimer's disease. These results show that administration of voriconazole, or other compound candidates herein, dramatically improves cognitive functions and alleviates AD pathology. This indicates that intervention with voriconazole, or other compound candidates herein, upon the diagnosis of AD or related tauopathies can display strong therapeutic effects.

While various embodiments have been described above, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the present application, and it is not intended to detail all those obvious modifications and variations of it which will become apparent to the skilled worker upon reading the description. It is intended, however, that all such obvious modifications and variations be included within the scope of the present application, which is defined by the following claims. The claims are intended to cover the components and steps in any sequence which is effective to meet the objectives there intended, unless the context specifically indicates the contrary.

1. A method of treating a neurodegenerative disease in a subject in need thereof comprising
administering to a subject in need thereof a therapeutically effective amount of a pharmaceutical composition comprising voriconazole, or analog compound of voriconazole, or variant of voriconazole with a generic substitution in its chemical structure, and a pharmaceutically acceptable carrier.

2. The method of claim 1, wherein the neurodegenerative disease is one or more selected from the group consisting of Alzheimer's disease and tauopathies.

3. The method of claim 1, wherein the voriconazole is packaged for delivery in a titratable dosage form.

4. The method of claim 1, wherein the voriconazole is packaged such that delivery is targeted to an area selected from the group consisting of: sublingual; buccal; parenteral; oral; rectal, nasal; and the pulmonary system.

5. The method of claim 1, wherein the voriconazole is in the form selected from the group consisting of: gel; gel spray; tablet; liquid; capsule and for vaporization.

6. The method of claim 1, further comprising administering a secondary neuroprotective therapeutic or adjunctive therapeutic agent.

7. The method of claim 6, wherein the secondary neuroprotective therapeutic agent or other adjunctive therapeutic agent is an CYP46A1 inhibiting agent selective for a glutamate receptor, an alkalinizing agent, an anticoagulant, tissue plasminogen activator, aspirin, or an anti-platelet agent.

8. The method of claim 6, wherein the secondary neuroprotective therapeutic or adjunctive therapeutic agent is administered to said subject in a coordinate administration protocol, simultaneously with, prior to, or after, administration of said voriconazole,

9. The method of claim 6, wherein the secondary neuroprotective therapeutic or adjunctive therapeutic agent is administered to said subject by a different method than the administration of the voriconazole.

10. The method of claim 2, wherein the neurodegenerative disease is Alzheimer's disease.

11. The method of claim 1, wherein said voriconazole is administered to said subject nasally, intrathecally, and/or epidurally.

12. A method of treating Alzheimer's disease in a subject in need thereof comprising
administering to a subject in need thereof a therapeutically effective amount of a pharmaceutical composition comprising voriconazole, or analog compound of voriconazole, or variant of voriconazole with a generic substitution in its chemical structure, and a pharmaceutically acceptable carrier.

13. The method of claim 12, wherein the Alzheimer's disease is at a stage of pre-clinical Alzheimer's disease.

14. The method of claim 12, wherein the voriconazole is packaged for delivery in a titratable dosage form.

15. The method of claim 12, wherein the voriconazole is packaged such that delivery is targeted to an area selected from the group consisting of: sublingual; buccal; parenteral; oral; rectal, nasal; and the pulmonary system.

16. A method of treating acute brain injury in a subject in need thereof, comprising:
administering to a subject in need thereof a therapeutically effective amount of a pharmaceutical composition comprising voriconazole, or analog compound of voriconazole, or variant of voriconazole with a generic substitution in its chemical structure, and a pharmaceutically acceptable carrier.

17. The method of claim 16, wherein the voriconazole is packaged for delivery in a titratable dosage form.

18. The method of claim 16 wherein said voriconazole is administered to said subject nasally, orally, and/or in liquid form.

19. The method of claim 16, wherein the acute brain injury is cerebral ischemia.

20. The method of claim 16, wherein the acute brain injury is brain trauma.
